(19) United States
(12) Patent Application Publication
(10) Pub. No.: US 2025/0107726 A1
(43) Pub. Date: Apr. 3, 2025

(54) AMBIENT NOISE MONITORING FOR HEARING TEST

(71) Applicant: Apple Inc., Cupertino, CA (US)

(72) Inventors: Jianjun HE, San Jose, CA (US); Aditya SRINIVAS, San Francisco, CA (US); Yue ZHAO, San Jose, CA (US); Zhangli CHEN, San Jose, CA (US)

(21) Appl. No.: 18/818,485

(22) Filed: Aug. 28, 2024

Related U.S. Application Data
(60) Provisional application No. 63/541,769, filed on Sep. 29, 2023.

(51) Int. Cl. A61B 5/12; G10K 11/16

(52) U.S. Cl. CPC A61B 5/121; G10K 11/16

(57) ABSTRACT

Aspects of the subject technology provide for, while attenuating noise from a physical environment at a first device, for example, by active noise cancellation, outputting, by the first device, an audio signal for detection by a user. Aspects of the subject technology also provide, by the first device and to a second device, an indication of an estimated noise level of the attenuated noise at the first device for storage in association with an indication of whether the audio signal was detected by the user.

### CROSS REFERENCE TO RELATED APPLICATIONS

This application claims the benefit of U.S. Provisional Application No. 63/541,769, entitled “Ambient Noise Monitoring For Hearing Test,” filed Sep. 29, 2023, the entirety of which is incorporated herein by reference.

### TECHNICAL FIELD

The present description relates generally to electronic devices, including, for example, noise cancellation for electronic devices.

### BACKGROUND

Headphones are an audio device that includes a pair of speakers, each of which is placed proximate to a user's ears. Over-the-ear or on-the-ear headphones include the pair of speakers connected to each other by a band which is worn on or around the user's head. Earphones or earbuds are headphones which include the pair of speakers inserted into the user's ears. The pair of speakers of the headphones are normally connected (e.g., wired or wirelessly connected) to a separate playback device, such as an MP3 player, that drives each of the speakers of the devices with an audio signal in order to produce sound (e.g., music). Some headphones can include audio input devices to monitor the ambient acoustical environment. Some headphones have the capability of generating an anti-noise signal to acoustically cancel or reduce the sound from the ambient acoustical environment.

### BRIEF DESCRIPTION OF THE DRAWINGS

Certain features of the subject technology are set forth in the appended claims. However, for purpose of explanation, several embodiments of the subject technology are set forth in the following figures.

FIG. 1 illustrates a diagram of an example electronic device that may implement aspects of the subject technology in accordance with one or more implementations.

FIG. 2 illustrates a diagram of another example electronic device that may implement aspects of the subject technology in accordance with one or more implementations.

FIG. 3 illustrates a block diagram of an audio signal processing architecture for an electronic device in accordance with one or more implementations.

FIG. 4 illustrates additional details that may be implemented in the audio signal processing architecture of FIG. 3 in accordance with one or more implementations.

FIG. 5 illustrates a flow diagram of an example process for administering at least a portion of a hearing test, in accordance with one or more implementations.

FIG. 6A illustrates a flow diagram for determining the estimated noise level, in accordance with some implementations.

FIG. 6B illustrates a flow diagram for determining the estimated noise level, in accordance with other implementations.

FIG. 7 illustrates an example auditory filter which can be used for evaluating the estimated noise, in accordance with some implementations.

FIG. 8 illustrates a flow diagram of an example process for a hearing test process, in accordance with some implementations.

FIG. 9 illustrates a flow diagram of an example process for calculating a noise interruption decision, in accordance with some implementations.

FIGS. 10A and 10B illustrate flow diagrams of an example process for performing a hearing test, in accordance with some implementations.

FIG. 11 illustrates a flow diagram of an example process for estimating a noise level of the ambient noise, in accordance with some implementations.

FIG. 12 illustrates a flow diagram of an example process for outputting an audio signal for detection by a user, in accordance with some implementations.

FIG. 13 illustrates a flow diagram of an example process for outputting an audio signal for detection by a user while actively attenuating noise from a physical environment at a first device, in accordance with some implementations.

FIG. 14 illustrates an example electronic system with which aspects of the subject technology may be implemented in accordance with one or more implementations.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components are shown in block diagram form in order to avoid obscuring the concepts of the subject technology.

Audio devices, such as headphones and/or earbuds can provide active noise cancellation (ANC) (e.g., in a noise cancellation mode of the audio devices) in which a microphone audio input device of the headphones and/or earbuds (and/or a connected electronic device) receives an audio signal including ambient noise from the environment around the headphones and/or earbuds, and the audio device generates an anti-noise signal that, when output by a speaker, cancels some or all of the ambient noise before the ambient noise is received in the ear canal of the user. Also, headphones and/or earbuds can provide passive noise cancellation (PNC) due to the physical interface between the headphones or earbuds and the ears. For example, headphones and earbuds can at least partially block ambient noise from the environment from entering the user's ear canal, thereby causing a reduction in ambient noise at the user's ear drum. The amount of PNC can vary based on the fit of the headphones or earbuds and the ear or ear canal.

Hearing tests can be given to a user wearing headphones in which various tones are played in the left ear and/or right ear of the user at different volumes. In a clinical setting, typically the user would go into a sound isolated environment and wear medical-grade lab-calibrated and/or lab-certified headphones designed for such tests. Alternatively, or in addition, users may be required to wear special insert headphones to block ambient noise. For example, these clinical hearing tests may be conducted in a quiet audiologist booth (<20 dBA) or may require a special insert headphone to block ambient noise. Clinical hearing tests may be too impractical, costly, or inconvenient for some individuals.

Aspects of the subject technology may provide an enhanced capability to conduct a hearing test, for example, in a non-clinical setting using consumer devices. This can result in more reliable non-clinical hearing tests which may be used for diagnostic results. This can be achieved by utilizing passive noise cancellation (PNC) and/or active noise cancellation (ANC) to reduce the ambient noise in the physical environment of the user that reaches the user's ears. In addition, a level, type, and/or interference of the remaining noise from the physical environment (i.e., the ambient noise which is not cancelled or reduced and reaches the user's eardrum) can be estimated or determined. Based on these estimates or determinations, actions may be taken within a hearing test procedure.

Some aspects of the subject technology may compare the estimated noise level of the remaining noise (i.e., after PNC or ANC) to a threshold. When a hearing test tone is being played and when the estimated noise level of the remaining noise is below the threshold, the test tone result is deemed valid. When a hearing test tone is being played and when the estimated noise level of the remaining noise is above the threshold, an indication is generated that indicates that the test tone result is invalid. In such cases, the test tone may be tested again. In some implementations, for example, where only PNC is being used, if the estimated noise level fails to meet the threshold, then ANC can also be used to further reduce the estimated noise level of the remaining noise (i.e., after PNC) to the meet the threshold.

Some aspects of the subject technology may determine the estimated level of the remaining noise based, at least in part, on ear fit of headphones or earbuds, for example by utilizing ear fit test data to determine how effective the active noise cancellation and/or passive noise cancellation is for the headphones or earbuds. Some aspects of the subject technology may determine a type of ambient noise as being stationary (e.g., continuous) noise or instantaneous noise and a noise level of ambient noise can be filtered as being A-weighted, octave band, or auditory band. Some aspects of the subject technology may estimate an interference of the remaining noise with the hearing test output signal. Based on the estimated interference, a different hearing test output signal may be used, the threshold may be adjusted, and/or the test may be delayed. The subject technology advantageously provides the ability to provide a hearing test while estimating noise in the ear canal which may interfere with the hearing test so that the result of the hearing test may be deemed more reliable than in hearing test processes which do not use noise estimates. In addition, the subject technology advantageously provides the ability to direct the hearing test based on characterizations of the estimated noise in the ear canal, for example by pausing the test, retesting a tone, or choosing a test tone based on the interference level at an auditory filter output. Further, the subject technology advantageously utilizes microphones and processing on headset and/or playback device to perform estimating of the noise in the ear canal and determining if the estimated noise would result in a distraction during a hearing test or interference with the hearing test.

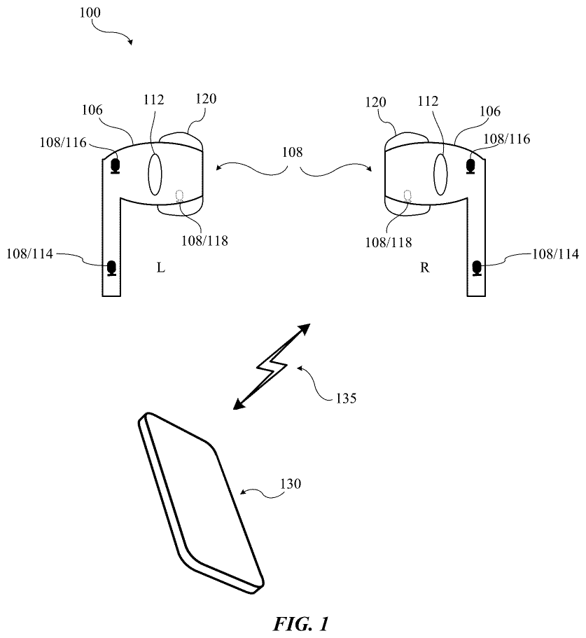

FIG. 1 illustrates an example electronic device in accordance with one or more implementations. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

In the example of FIG. 1, electronic device 100 has been implemented using a housing 106 that is sufficiently small to be portable and carried or worn by a user. Specifically, FIG. 1 illustrates an example of the electronic device 100 implemented as an earbud or pair of earbuds. In this example, the housing 106 is shaped for seating in the user's concha and/or aperture of the ear canal and for interfacing with the user's ear canal. An optional sealing member 120 may interface with the housing 106 to provide a sealing effect between the electronic device 100 and the user's ear canal. The sealing member 120 may be made of any suitable material, such as silicone, plastic, rubber, foam, a polymer, a composite thereof, or combinations thereof.

In one or more other implementations, the earbud of FIG. 1 may be used in conjunction with another electronic device 130, such as a smartphone or tablet computer to which microphone signals received by microphones 114, 116, and/or are transmitted and/or from which audio output signals for the speaker 112 are received. In one or more implementations, the earbud of FIG. 1 may include processing circuitry that performs one or more of the operations described herein.

In the example of FIG. 1, housing 106 includes openings 108. For example, openings 108 may form one or more respective ports for one or more respective audio components. In the example of FIG. 1, the electronic device 100 includes an opening 108 that forms a speaker port for a speaker 112 disposed within the housing 106, another opening 108 that forms a microphone port for a microphone 114 disposed within the housing 106, another opening 108 that forms a microphone port for a microphone 116 disposed within the housing 106, another opening 108 that forms a microphone port for a microphone 118 disposed within the housing 106. The openings 108 for the microphones 114 and 116 are disposed on an external surface of the housing 106 and the opening 108 for the microphone 118 is disposed on an internal surface of the housing 106 such that it is disposed within the opening 108 for the speaker 112.

In various implementations, the housing may also include fewer openings or additional openings. Additional openings may include openings for one or more additional audio input devices (e.g., microphones), one or more pressure sensors, one or more light sources, one or more light sensors, or other components that receive or provide signals from or to the environment external to the housing 106. Openings such as openings 108 may be open ports or may be completely or partially covered with a permeable membrane or a mesh structure that allows air and/or sound to pass through the openings.

Although the several openings 108 described above are shown in FIG. 1, this is merely illustrative. For example, one or more of the microphones 114, 116, and 118 may be omitted. One opening 108, two openings 108, or more than two openings 108 may be provided on the one or more sidewalls of the housing 106, on a rear surface of housing 106, a front surface of housing 106, and/or an inside surface of the housing 106. In some implementations, one or more groups of openings 108 in housing 106 may be aligned with a single port of an audio component within housing 106. Housing 106, which may sometimes be referred to as a case, may be formed of plastic, glass, ceramics, fiber composites, metal (e.g., stainless steel, aluminum, etc.), other suitable materials, or a combination of any two or more of these materials. The electronic device 100 also includes additional components such as processing circuitry (e.g., one or more processors), memory, a power source such as a battery, communications circuitry, and the like.

In one or more use cases, one or more of the speakers 112 may generate a speaker output based, for example, on a downlink communications signal or a device-generated or streaming audio signal. In one or more implementations, the speaker(s) 112 may be driven by an output downlink signal that includes far-end acoustic signal components from a remote device, such as the device 130. In one or more use cases, while a near-end user is using the electronic device 100 to input and/or transmit their own speech, ambient noise surrounding the user may also be present in the environment around the electronic device 100. Thus, the microphones 114 and 116 may capture the user's own speech as well as the ambient sounds around the electronic device 100.

Aspects of the subject technology described herein may be performed by one or more processors of the electronic device 100 of FIG. 1 and/or may be performed by a processor inside the electronic device 130, upon receiving the microphone signals from a wired or wireless data communication link 135 with the electronic device 100. The electronic device 100 in the example of FIG. 1 may include communications circuitry for communicating with one or more other electronic devices 130 via the wired or wireless data communication link 135. In use, microphones 114, 116, and/or 118 in the earbud may receive ambient sounds in the physical environment of the electronic device 100. The earbuds constituting the electronic device 100 of FIG. 1 may include a left earbud L and a right earbud R for use in a user's left and right ears, respectively.

In the example of FIG. 1, the electronic device 100 includes a speaker 112, a top microphone (e.g., microphone 116) whose sound sensitive surface faces a direction that is opposite the eardrum of the user when the earbud is worn, a bottom microphone (e.g., microphone 114) that is located in or near an end portion of the housing 106 of the earbud where it is the closest microphone to the user's mouth, and an error microphone 118 that senses the sound at or near the user's eardrum (e.g., in the user's ear canal). In the example of FIG. 1, the error microphone 118 may be in a position and orientation to receive an output from the speaker 112 and/or one or more ambient sounds (e.g., ambient noise, voices of people other than the user of the electronic device 100, a voice of the user of the electronic device 100, or the like) that leak past the housing 106 and/or sealing member 120 to reach the error microphone 118.

In one or more implementations, the top and bottom microphones of FIG. 1 can be used as, or as part of, a microphone array for purposes of pickup beamforming. More specifically, the microphone arrays may be used to create microphone array beams which can be steered to a given direction by emphasizing and deemphasizing selected top and bottom microphones (e.g., to enhance pick up of the user's voice from the direction of their mouth or another sound in the physical environment). Similarly, the microphone array beamforming can also be configured to exhibit or provide pickup nulls in other given directions, to suppress pickup of an ambient noise source. Accordingly, the beamforming process, also referred to as spatial filtering, may be a signal processing technique using the microphone array for directional sound reception.

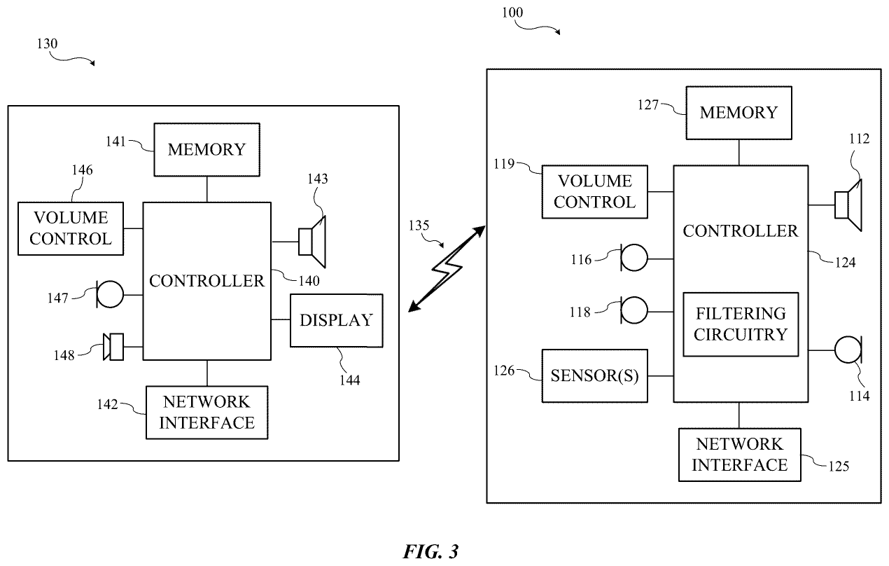

In one or more implementations, when the electronic device 100 is implemented as earbuds as in FIG. 1, the electronic device 100 may include a battery device, one or more processors, and/or a communication interface (see FIG. 3). The processors of the earbud may be a digital signal processing chip that processes an audio signal(s) (e.g., microphone signal(s)) from at least one of the microphones 114, 116, and/or 118. The communication interface may include a Bluetooth™ receiver and transmitter to communicate acoustic signals from the microphones 114, 116, and/or 118 in both directions (uplink and downlink), with an external device 130 such as a smartphone, a tablet computer, a laptop computer, or a desktop computer, in some implementations. Further, the earbuds may be wired earbuds or untethered wireless earbuds that communicate with each other and with the external device 130 via Bluetooth™M signals.

In some aspects, the electronic device 100 may be communicatively coupled to the electronic device 100, such as demonstrated above via a wireless connection. In other aspects, the electronic device 130 may be communicatively coupled with the electronic device 100 via other methods. For example, both devices may couple via a wired connection. In this case, one end of the wired connection may be (e.g., fixedly) connected to the electronic device 100, while another end may have a connector, such as a media jack or a universal serial bus (USB) connector, which plugs into a socket of the electronic device 130. Once connected, the electronic device 130 may be configured to drive one or more speakers of the electronic device 100 with one or more audio signals, for example, for a hearing test, via the wired connection. For instance, the electronic device 130 may transmit the audio signals as digital audio (e.g., PCM digital audio). In another aspect, the audio may be transmitted in analog format.

In another aspect, the electronic device 130 may instruct the electronic device 100 to generate, for example, via an on-device processor, an audio signal corresponding to an audio signal for a hearing test. The audio signal may be generated by the electronic device 100 and played on one or more speakers 112 of the electronic device 100.

As is discussed in further detail below, microphones 114, 116, and/or 118, and/or other microphones and/or sensors of the electronic device 100 may be used, in conjunction with the architectures/components described herein, for automatic noise cancellation. In addition, the one or more processors of the electronic device 100 and/or the electronic device 130 may be used to estimate and/or characterize the remaining noise after noise cancellation which is estimated to reach the user's eardrum. Based on the estimation of the remaining noise and/or characterization of the remaining noise a hearing test can be evaluated or altered.

In some aspects, the various processes described herein can make use of a device monitoring process to direct the selection and use of various microphones described herein. For example, the noise estimation and/or characterization of the remaining noise after noise cancellation can be performed utilizing a microphone which is selected as having the most appropriate device response of the available microphones. For example, the electronic device 100 may have a left side and a right side, with each side having multiple microphones available for use, such as described herein. In such implementations, a separate device monitoring process can monitor and maintain which of such microphones exhibit good characteristics for usage during the noise estimation process. For example, one or more microphones may have occlusion from dirt or debris, water infiltration, or physical problems which would make such microphone undesirable for use. Therefore, the noise estimation process can utilize the most appropriate microphone of the left-side or the right-side component (e.g., bud) of the electronic device 100, as determined by a device monitoring process, for performing the noise estimation according to the processes described herein.

Similarly, occlusion or hardware issues can negatively impact ANC when the microphone used for ANC is broken or occluded. Thus, aspects can compute the drop or effect of such issues on ANC attenuation. For example, even if a separate process for selecting a better microphone is not used, the impact of occlusion or hardware defects in the ANC microphone can be used to help in the estimation of the noise level remaining. Or, in some aspects, the ANC can be adjusted in accordance with the estimated impact on ANC due to an occluded or defective microphone.

Also, occlusion or hardware issues can negatively impact noise estimation when the microphone used for noise estimation is broken or occluded. Thus, aspects can take into account the drop or effect of such issues on noise estimation. For example, even if a separate process for selecting a better microphone for noise estimation is not used, the impact of occlusion or hardware defects in the noise estimation can be accounted for, for example, by providing equalization adjustments on the microphone to reduce the impact of occlusion or detected hardware issues.

In another aspect, prior to the use of microphone inputs for the purposes of ANC and/or noise level estimation, a noise reduction process can be performed on the microphone signal to reduce the self-noise of the microphone. Reducing the self-noise of the microphone, for example, provides that the associated microphone captures a more accurate level of ambient noise, e.g., for the purpose of ANC or noise estimation.

The electronic device 130 may be any electronic device (e.g., with electronic components, such as one or more processors, memory, etc.) that is capable of streaming audio content, in any format, such as stereo audio signals, for playback (e.g., via one or more speakers integrated within the source device and/or via one or more output devices, as described herein). For example, the source device may be a desktop computer, a laptop computer, a digital media player, etc. In one aspect, the device may be a portable electronic device (e.g., being handheld operable), such as a tablet computer, a smart phone, etc. In another aspect, the source device may be a wearable device (e.g., a device that is designed to be worn on (e.g., attached to clothing and/or a body of) a user, such as a smart watch. In some aspects, the electronic device 130 may include an interface and programming contained in memory which when executed by the one or more processors performs a hearing test utilizing the electronic device 100.

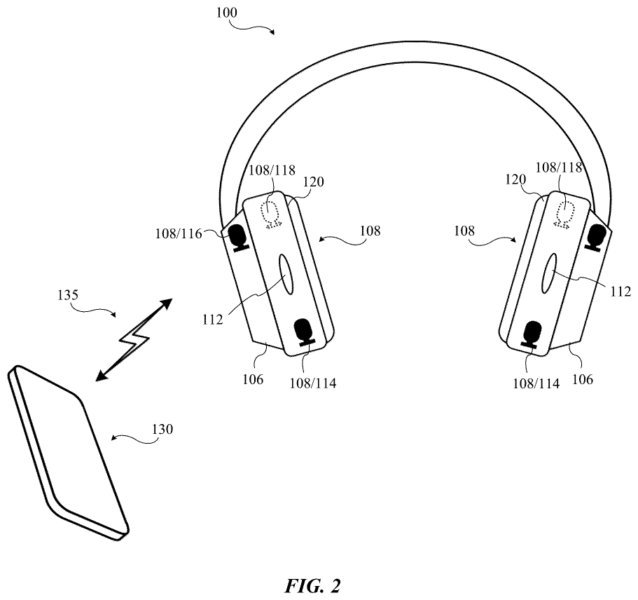

Referring now to FIG. 2, although an example is shown in FIG. 1 in which the electronic device 100 is implemented as earbuds, in other implementations, such as that illustrated in FIG. 2, the electronic device 100 may be implemented as headphones including a housing 106 that includes a pair of earcups that are configured to be placed over or on the user's ears. Further, the headphones may be wired or untethered wireless headphones that communicate with each other by a wire or wirelessly and with an external device such as a smartphone or a tablet computer via Bluetooth™ signals.

The headphones of FIG. 2 may include openings 108 for speakers 112 and one or more openings 108 for one or more microphones 114, 116, and 118, as described above, which descriptions are not repeated. In addition, a sealing member 120 may both cushion the earcups of the headphones and provide sealing between the housing 106 and the user's ear from the ambient environment. The sealing member 120 may be made of any suitable material, such as silicone, plastic, rubber, foam, a polymer, fabric, a composite thereof, or combinations thereof.

FIG. 3 shows a block diagram of the electronic device 130 and of the electronic device 100 that are wirelessly coupled together according to one aspect. The electronic device 130 includes a controller 140, memory 141, a network interface 142, a speaker 143, a display 144, a microphone 147, a camera 148, and a volume control 146. In one aspect, the source device may include more or less elements as described herein. For instance, the device may include several speakers and/or microphones, or may not include the camera or the display.

The controller 140 may be a special-purpose processor such as an application-specific integrated circuit (ASIC), a general purpose microprocessor, a field-programmable gate array (FPGA), a digital signal controller, or a set of hardware logic structures (e.g., filters, arithmetic logic units, and dedicated state machines). The controller 140 is configured to perform operations for conducting a hearing test, audio signal processing operations, and/or networking operations. More about the operations that may be performed by the controller 140 are described herein.

The memory 141 may be any type of non-transitory machine-readable storage medium. Examples may include read-only memory, random-access memory, CD-ROMS, DVDs, magnetic tape, optical data storage devices, flash memory devices, and phase change memory.

The camera 148 may be a complementary metal-oxide-semiconductor (CMOS) image sensor that is capable of capturing digital images including image data that represent a field of view of the camera, where the field of view includes a scene of an environment in which the electronic device 130 is located. In some aspects, the camera may be a charged-coupled device (CCD) camera type. The camera is configured to capture still digital images and/or video that is represented by a series of digital images. In one aspect, the camera may be positioned anywhere about/on the electronic device 130. In some aspects, the electronic device 130 may include multiple cameras (e.g., where each camera may have a different field of view).

The microphone 147 may be any type of microphone (e.g., a differential pressure gradient micro-electro-mechanical system (MEMS) microphone) that is configured to convert acoustical energy caused by sound wave propagating in an acoustic environment into a microphone signal. In some aspects, the microphone may be an “external” (or reference) microphone that is arranged to capture sound from the acoustic environment.

The speaker 143 may be an electrodynamic driver that may be specifically designed for sound output at certain frequency bands, such as a woofer, tweeter, or midrange driver, for example. In one aspect, the speaker 143 may be a “full-range” (or “full-band”) electrodynamic driver that reproduces as much of an audible frequency range as possible.

The display 144 is designed to present (or display) digital images or videos of video (or image) data. In one aspect, the display may use liquid crystal display (LCD) technology, light emitting polymer display (LPD) technology, or light emitting diode (LED) technology, although other display technologies may be used in other aspects. In some aspects, the display may be a touch-sensitive display screen that is configured to sense user input as touches or taps on the screen, and in response produce one or more control signals. In some aspects, the display may use any touch sensing technologies, including but not limited to capacitive, resistive, infrared, and surface acoustic wave technologies.

The volume control 146 is configured to adjust a volume level of sound output of the electronic device 130 (and/or the electronic device 100) in response to receiving a user-adjustment (e.g., user input) at the control. In one aspect, the volume control may be a “master” volume control that is configured to control the overall volume level (e.g., sound output level of the speaker 143) of the electronic device 130. In another aspect, when the electronic device 130 is communicatively coupled with the electronic device 100 in order to stream audio content to the electronic device 100, for playback, the volume control may control the overall volume level of the electronic device 100 (as well). In one aspect, the volume level output on the electronic device 100 for a hearing test process or application may override the setting provided by way of the volume control 146, for example, by a user. In one aspect, the volume control may be a “physical” volume control that may be a dedicated volume input control, such as one or more buttons, a rotatable knob, or a physical slider. For instance, the volume control 146 may include at least two buttons, a “volume up” button, which may be configured to perform a stepwise increase to the volume each time it is pressed by the user, and a “volume down” button that may be configured to perform a stepwise decrease to the volume each time it is pressed by the user. In some aspects, the volume control may be any type of physical input device that can adjust the volume level.

According to one aspect, the electronic device 100 includes a controller 124, a network interface 125, an audio output device, such as speaker 112, three microphones 114, 116, and 118, one or more (other) sensors 126, a volume control 119, and memory 127. In one aspect, the device may include more or less elements. For example, the electronic device 100 may include one or more speakers, and/or may include one or more microphones. In one aspect, microphone 114 may be an input microphone, such as a vocal microphone, microphone 116 may be a reference microphone, and microphone 118 may be an error microphone, as described herein. In another aspect, the output device may not include an error microphone, or may include at least one reference microphone and at least one error microphone. For example, in the case of an earbuds or headphones device, an error microphone may sense sound inside the user's ear when the electronic device 100 is positioned on (or in) the user's ear.

The sensors 126 may include one or more other sensors that are designed to produce sensor data. For instance, the sensors may include an IMU or a proximity sensor. In which case, the IMU may be designed to produce motion data that indicates (changes in) the position and/or orientation of the output device. In another example, the sensors may include an accelerometer that may be arranged and configured to receive (detect or sense) speech vibrations that are produced while a user (e.g., who may be wearing the output device) is speaking, and produce an accelerometer signal that represents (or contains) the speech vibrations. Specifically, the accelerometer is configured to sense bone conduction vibrations that are transmitted from the vocal cords of the user to the user's ear (ear canal), while speaking and/or humming. For example, when the electronic device is a wireless headset, the accelerometer may be positioned anywhere on or within the headphone, which may touch a portion of the user's body in order to sense vibrations.

In one aspect, controller 124 may be configured to perform audio signal output and noise estimation operations, (other) audio signal processing operations and/or networking operations, as described herein. For instance, the controller may be configured to obtain (or receive) audio data (as an analog or digital audio signal) that includes audio content, such as music or test tones for playback through the speaker 112. In some aspects, the controller may obtain audio data from memory 127, or the controller may obtain audio data from another device, such as the source device via the network interface 125. For instance, the output device may stream an audio signal from the source device (e.g., via the Bluetooth™ connection) for playback through the speaker 112. The audio signal may be a signal input audio channel (e.g., mono). In another aspect, the controller may obtain two or more input audio channels (e.g., stereo) for output through two or more speakers. In one aspect, in the case in which the output device includes two or more speakers, the controller may perform additional audio signal processing operations.

In one aspect, the volume control 119 may perform similar operations as the volume control 146 of the source device. For instance, upon receiving user input, the control 119 may adjust the (e.g., overall) volume of sound output by the (e.g., speaker 112 of the) electronic device 100. In some aspects, the volume control 119 may be used to adjust the volume setting at the electronic device 130.

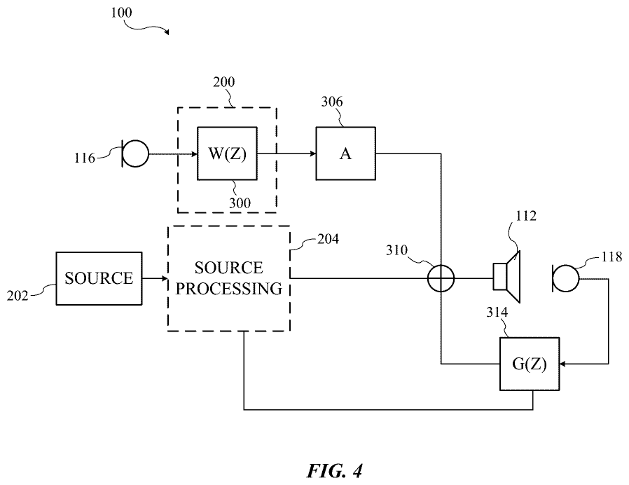

FIG. 4 illustrates details for active noise cancellation that may be included in the electronic device 100, such as earbuds or headphones, for example of FIG. 3. As shown in FIG. 4, filtering circuitry 200 may include an active noise cancellation (ANC) filter 300 operating in a noise cancellation mode of the electronic device 100. As shown, the ANC filter 300 may generate the noise cancellation signal, and provide the noise cancellation signal to a gain stage 306. In one or more implementations, the ANC filter 300 may utilize, for example, at the filtering circuitry 200 provided by another process to select the better performing microphone 116 between a left-side component and a right-side component of the electronic device 100. In one or more implementations, when occlusion or hardware defect is detected in the microphone 116, the filtering circuitry 200 can provide compensation for the ANC and/or equalization settings to the microphone 116. In one or more implementations, the ANC filter 300 may include a variation of an optimal filter that can produce an estimate of the ambient sounds by filtering a microphone signal corresponding to the microphone 116 (e.g., an audio signal received directly from the microphone 116 or an audio signal received by the microphone 116 and pre-processed prior to providing the audio signal to the filtering circuitry 200) and can generate a noise cancellation signal (e.g., an anti-noise signal) corresponding to that estimate. For example, an estimate of the ambient sound(s) and a corresponding noise cancellation signal (e.g., anti-noise signal) can be produced, for example, by adaptive prediction and/or by using a prediction filter that exploits a low-pass characteristic of the audio signal. The noise cancellation signal generated by the ANC filter 300 may be adjusted by the gain stage 306 and combined with an audio signal by the adder 310, for output by the speaker 112. In one or more implementations, the ANC filter 300 can be implemented at least partially in hardware, firmware or software.

As shown in FIG. 4, the gain stage 306 may apply a gain, A, to the noise cancellation signal to provide positive or negative adjustment to the noise cancellation signal. In this way, the applied gain can help control the eardrum sound pressure level at or near the eardrum of the user of the electronic device 100.

FIG. 4 also illustrates other aspects of the circuitry that can be provided to generate the noise cancellation signal from the ANC filter 300. As illustrated in FIG. 4, the ANC filter 300 may generate a noise cancellation (e.g., anti-noise) signal that, when output by the speaker 112, destructively interferes with ambient sound that leaks past the housing 106 (and past the sealing member 120) (i.e., noise remaining after the passive noise cancellation aspects of the electronic device 100) and into the user's ear canal. The leaked ambient noise and the noise cancellation portion of the output of the speaker 112 (based on some or all of the noise cancellation signal generated by the ANC filter 300) may be combined acoustically in the user's ear canal, intentionally in a destructive manner so as to result in a very small residual noise or error. As shown, the error microphone 118 may receive this residual noise or error, in addition to any source information, that is being simultaneously output by the speaker 112, and may provide an error signal to a feedback noise filter 314. As shown, in one or more implementations, the feedback noise filter 314 may generate an anti-residual noise signal that can be mixed into the mixed output signal by the adder 310. The feedback noise filter 314 can base the anti-residual noise signal in part on the source information from source processing block 204, for example, by subtracting the source information from the error microphone 118 data to find the residual noise or error.

The audio signal from source 202 (e.g., electronic device 130) may be received and processed by a source processing block 204. The source processing block 204 can analyze the audio source and determine what tone or frequencies are being received for playback. In the case of a hearing test, the audio signal is one of a series of hearing test tones. For example, the audio signal may be a 1 kHz tone signal. In a hearing test, the level or gain of the tone can be altered or normalized to override volume settings of the electronic device 100 and/or electronic device 130 so that a specified level is output on the speaker 112 of the electronic device 100. Additional aspects of the source processing block 204 are discussed in further detail below.

Other features of the electronic device 130 may be implemented which are not shown. For example, the electronic device 130 can include a pass-through audio filter to generate a pass-through audio signal to receive and amplify ambient noise, in some operative modes. As another example, the ANC filter 300 may be adaptively controlled to adjust the adaptive filter and/or gain 306 to optimize the ANC filter 300 coefficients to seek to minimize residual noise or error.

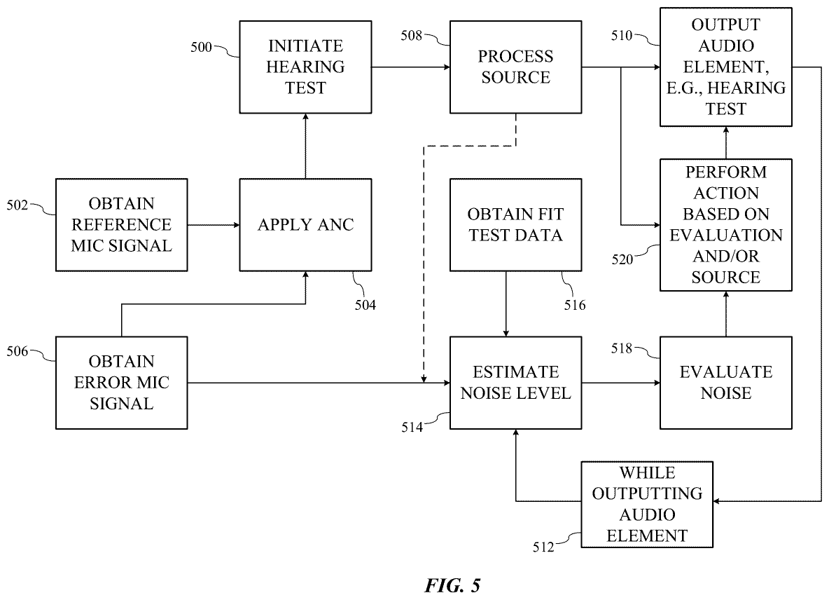

FIG. 5 illustrates a flow diagram of an example process for administering at least a portion of a hearing test. Although FIG. 5 is discussed with respect to a hearing test, it should be understood that the flow of FIG. 5 may be applied to any type of playback situation. For example, in a hearing test, an audio element played is a portion of a hearing test, however, the audio element could be another type of audio source. One or more blocks (or operations) of the process flow diagram of FIG. 5 may be performed by one or more other components and other suitable devices. Further for explanatory purposes, the blocks are described herein as occurring in serial, or linearly. However, multiple blocks of the process may occur in parallel. In addition, the blocks of the process need not be performed in the order shown and/or one or more blocks of the process need not be performed and/or can be replaced by other operations.

At block 500, a device (e.g., electronic device 100) is positioned on or in a user's ear. The position of the device on or in the user's ear may provide passive noise cancellation (PNC), e.g., by blocking some of the ambient noise from entering the user's ear canal, though some of the noise will leak past the device and enter the user's ear. A sealing member, such as the sealing member 120, may be used to further enhance the effectiveness of the device on or in the user's ear to block more of the ambient noise from entering the user's ear canal.

A hearing test can be initiated, which will be administered on and/or by the device. The hearing test can be initiated from another user device, such as the electronic device 130. For example, a user can launch an application on the user device for administering hearing tests. The user can begin the test using the application.

At block 502, the device, such as electronic device 100, may obtain a reference mic signal, such as from the reference mic 116. The reference mic signal picks up the ambient noise in the physical environment of the device. The reference mic signal may be used throughout the flow of conducting the hearing test.

At block 504, the device (e.g., electronic device 100) may be used to apply active noise cancellation (ANC) in a noise cancellation mode of the device to output an anti-noise signal on speaker(s), such as speaker 112, of the device. The anti-noise signal combines with the noise leaking by the device and/or sealing member of the device to attenuate or cancel out at least some of the noise leaking by the device and/or sealing member. The ANC process is described above with respect to FIG. 4 and is not repeated. The ANC process may be used throughout the flow of conducting the hearing test. In some implementations, applying ANC may not be performed and instead only PNC is performed.

At block 506, in some implementations an error mic signal may be obtained by the device (e.g., electronic device 100) by an error mic, such as error mic 118. The error mic signal receives feedback which indicates the effectiveness of the ANC and/or PNC. The error mic signal can be used to generate a further anti-noise signal (e.g., to combine with the anti-noise signal from block 504) to further attenuate the remaining noise in the ear canal. In some implementations, the error mic signal can also or instead be used as input to the ANC block 504 to provide better attenuation or noise cancellation based on the reference mic signal from block 502, for example, by altering the gain of the anti-noise signal generated from the reference mic signal.

At block 508, a source input is processed. In some implementations, the source input may be an audio signal that is provided from the user device (e.g., electronic device 130), such as the user's phone, tablet, computer, and so forth. In other aspects, the source input may be an audio signal instruction to generate and play a certain sound, such as a hearing test tone. In such aspects, the on-device processor or controller can be used to generate the sound and provide it to the speaker(s), such as speaker 112. In some implementations, for example where a hearing test is not being conducted but some other media is being played, the source input may be a media element which distraction-free playback is desired. For example, the source input may be a voicemail message, a podcast, a news story, an audiobook, and so forth. In some implementations, for example where a hearing test is being conducted, the source input may correspond to a hearing test tone to be played in the right ear, left ear, or both ears of the device. As such, the source input for the hearing test tone may be an audio signal provided from a user device, such as the electronic device 130, for playback on the device (e.g., electronic device 100) or may be an instruction from the user device to the device (e.g., electronic device 100) to generate and playback the test tone.

At block 510, the audio element (e.g., hearing test tone or media element) may be output on the device (e.g., electronic device 100). Thus, while the ANC is still being applied from block 504 (in such implementations), the device combines the output of the audio element with the anti-noise signal(s) from the ANC and outputs them on one or more speakers, such as speaker 112, of the device. In some aspects, the source from block 508 may be provided to another block 520, discussed in further detail below. In some implementations, the source from block 508 may be provided to another block 514, discussed in further detail below.

In some implementations, the audio element may not yet be output, and the remaining blocks executed prior to outputting the audio element.

At block 512, while outputting the audio element, at block 514, the noise level may be estimated. The estimated noise level indicates an estimate of the ambient noise that reaches the ear drum after leaking past the device and/or sealing member (e.g., after PNC) and after ANC (when utilized). The estimated noise level may be based on the reference mic signal, the error mic signal, and/or fit test data. The estimated noise level may be determined prior to outputting the audio element, in accordance with some aspects. In one or more implementations, the estimated noise level may utilize another process to select the better performing microphone of the available microphones of a left-side component and a right-side component of the electronic device 100. In one or more implementations, when occlusion or hardware defect is detected in a microphone used for estimating noise level compensation and/or equalization settings may be applied to the input signal from the microphone to reduce error associated with the occlusion or hardware defect. In one or more implementations, a process may also be performed on the input signal from the reference mic signal or the error mic signal to reduce the self-noise of the microphone.

Turning to block 516, fit test data may be obtained by the device (e.g., electronic device 100) from a user device (e.g., electronic device 130). The fit test data may indicate an effectiveness or performance of the housing (e.g., housing 106) and/or sealing member (e.g., sealing member 120) in providing PNC. In some implementations, a fit test may be conducted by the user device immediately prior to starting the hearing test, such as at block 500. In such aspects, the user, for example, can insert the device into the user's ears to engage the housing and/or sealing member of the device at the aperture of the ear canal. Then, upon starting the hearing test, prior to playing any hearing test tone, a system process or secondary application may be launched to conduct an ear fit test. The ear fit test can indicate a quality of the passive noise cancellation (i.e., blocking) provided by the housing or sealing member of the device.

In other aspects, previous fit test data which is stored in memory of the user device, such as electronic device 130, or in memory of the device, such as electronic device 100, may be obtained from the user device or the device. In other aspects, if the previous fit test data is older than a threshold period of time, such as 30 days, the user may be prompted to run an ear fit test.

It is noted that the ear fit test is optional. In some implementations, the ear fit test can be estimated or can be estimated based on generalized device data. For example, statistics can be kept on ear fit test data across a broad range of users and devices and the statistics can be used to estimate ear fit test data for the particular user in the present case.

In some aspects, the user device, such as electronic device 130, can provide the fit test data to the device, such as electronic device 100 to perform noise level estimation. In other aspects, the user device can utilize the fit test data to perform noise level estimation.

Returning to block 514, the estimated noise level can be determined by utilizing the optional fit test data, the optional error mic signal, and the reference mic signal. For example, the reference mic signal can be used to evaluate the ambient noise level of the physical environment of the device.

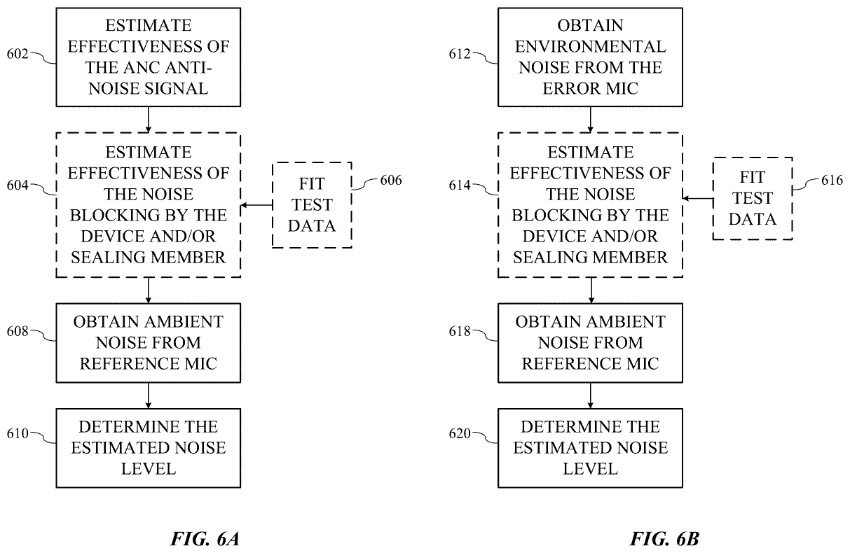

FIG. 6A illustrates a flow diagram for determining the estimated noise level, in accordance with some implementations. The flow diagram of FIG. 6A may be executed by the device, such as the electronic device 100, the user device, such as the electronic device 130, or a combination of the two. In block 602, the effectiveness of the ANC anti-noise signal (if ANC is utilized) is estimated. As noted above, the estimation may make use of an appropriate microphone on the left-side or right-side component of the electronic device, such as may be determined by a separate device monitoring process to monitor and select microphones on the electronic device for use. The estimation may also, or instead, apply equalization filters to the input signal or compensation measures to reduce error associated with a hardware defect and/or occlusion. A process may also be used to reduce self-noise generated by the microphone(s) used for noise estimation. In block 604, the effectiveness or performance of the PNC (e.g., noise blocking) by the device and/or sealing member may be estimated. In block 606, the ambient noise from the reference mic is obtained. In block 610, the estimated noise level is determined by comparing the output of block 602 and block 604 with the ambient noise 608. For example, if the reference mic signal indicates an input signal in the ambient environment of 60 dB and the ANC anti-noise signal is estimated to reduce the input signal to 35 dB and the noise blocking by the device and/or sealing member is estimated to reduce the input signal by 15 dB, then the estimated noise level of the noise reaching the ear drum may be estimated to be about 46 dB (e.g., using nomogram addition). In some aspects, at block 606, the fit test data can be provided and used at block 604 to more accurately estimate the noise reducing impact of the device and/or sealing member.

FIG. 6B illustrates a flow diagram for determining the estimated noise level, in accordance with other implementations. The flow diagram of FIG. 6B may be executed by the device, such as the electronic device 100, the user device, such as the electronic device 130, or a combination of the two. In some aspects, the error mic signal can be used to provide a characterization of the remaining noise which is present near the speaker of the device. As the error mic signal is disposed between the speaker of the device and the user's ear drum, the error mic signal can be used on its own to estimate the noise level of the noise remaining at the user's ear drum. As noted above, the estimation may make use of an appropriate microphone on the left-side or right-side component of the electronic device, such as may be determined by a separate device monitoring process to monitor and select microphones on the electronic device for use. The estimation may also, or instead, apply equalization filters to the input signal or compensation measures to reduce error associated with a hardware defect and/or occlusion. A process may also be used to reduce self-noise generated by the microphone(s) used for noise estimation. At block 612, the environmental noise from the error mic is obtained. As indicated in FIG. 5, the audio signal source can be provided to the block 514. As such, the audio signal source can be subtracted from the error mic signal to obtain the environmental noise. At block 620, the estimated noise level may be estimated from the environmental noise obtained by the error mic. In some aspects, the estimated noise level may be determined to be the environmental noise obtained by the error mic.

In other aspects, in block 614, the effectiveness or performance of the noise blocking by the device and/or sealing member may be estimated. In block 620, the estimated noise level may be estimated by combining the environmental noise obtained by the error mic and the estimated effectiveness or performance of the noise blocking by the device and/or sealing member. For example, if the environmental noise is estimated to be 35 dB and the effectiveness or performance of the blocking by the device and/or sealing member is estimated to add no more than 6 dB, then the estimated noise level may be determined to be no more than about 41 dB. In some aspects, at block 616, the fit test data can be provided and then used at block 614 to more accurately estimate the noise reducing impact of the device and/or sealing member.

In some aspects, in block 618, the reference mic signal can be used to obtain ambient noise in the physical environment of the device. Then, in block 620, the estimated noise level may be determined by utilizing the environmental noise obtained in block 612, the effectiveness or performance of the noise blocking by the device and/or sealing member estimated in block 614, and the ambient noise obtained in block 618. For example, the ambient noise can be combined with the estimated effectiveness or performance of the noise blocking by the device and/or sealing member to determine an estimated noise entering the ear canal which is not cancelled by ANC, which can then be combined with the environmental noise to provide the estimated noise level. In some aspects, at block 616, the fit test data can be provided and then used at block 614 to more accurately estimate the noise reducing impact of the device and/or sealing member.

In some aspects pertaining to each of the flow diagrams represented by FIGS. 6A and 6B, the noise estimation may make use of individual noise estimations determined from each side of the user, e.g., from both the left-side component and right-side component of the electronic device using one or more microphones from each of the left-side component and the right-side component. That is, a noise estimation level may be made for the left-side component and a noise-estimation may be made for the right-side component, for example, using any of the processes described herein. In such implementations, the final estimated noise level may be determined by combining the estimated noise levels from each of the left-side component and the right-side component to arrive at a single estimated noise level. Combining the noise levels can take into account which of the estimated noise levels is higher as well as which of the left-side or right-side components is playing a tone for a hearing test. Providing a single level as the noise estimation level can simplify further processing utilizing the estimated noise level, such as described below.

Returning to FIG. 5, after estimating the noise level at block 514, the estimated noise can be evaluated at block 518. In some aspects, evaluation of the estimated noise can include comparing the estimated noise to a predefined threshold. In some aspects, evaluation of the estimated noise may include performing frequency analysis or filtering on the estimated noise. In some aspects, evaluation of the estimated noise may include characterizing the estimated noise, including for example characterizing the estimated noise type as corresponding to a stationary noise or an instantaneous noise and/or characterizing the estimated noise level by A-weighted filtering, octave band filtering, or auditory filtering, or combinations thereof. In some aspects, evaluation of the estimated noise may include setting a threshold based on the audio signal source from block 508 and the characterization of the estimated noise and comparing the estimated noise to the threshold. The evaluation of the estimated noise will be discussed in greater detail below.

At block 520, an action can be performed based on the evaluation and/or audio signal source. For example, if the estimated noise fails to meet a threshold, if not used, ANC can be used to meet the threshold, or if used, ANC can be adjusted to meet the threshold. In another example, if the estimated noise meets a threshold, an indication can be stored that the estimated noise met the threshold. In another example, if the auditory filter output of the noise is determined to mask the audio signal source, then an indication can be stored that the estimated noise masked the audio signal source. In another example, if an instantaneous sound is observed which meets a threshold, then an indication may be stored that the instantaneous sound met the threshold. In another example, if a stationary sound is observed which meets a threshold, then an indication may be stored that the stationary sound met the threshold. In another example, if the estimated noise meets a threshold, outputting the audio signal source can be delayed until the estimated noise does not meet the threshold. In another example, prior to outputting the audio signal source, if the auditory filter output of the estimated noise level of the attenuated noise from the physical environment is determined to mask the audio signal source to be outputted, then the audio signal source can be changed to a different audio signal test source having a different frequency value which would not be masked by the frequency characteristics of the estimated noise. In another example, prior to outputting the source, the auditory filter output of the estimated noise can be used to determine an audio signal test source which would not be masked by the frequency characteristics of the estimated noise. In another example, prior to outputting the audio signal source, if an intensity or energy level of the estimated noise level of the attenuated noise from the physical environment is determined to cause an interference with the audio signal source to be outputted, then the audio signal source can be changed to a different audio signal test source having a different frequency value which would not have interference with the estimated noise level.

As noted above, the estimated noise level at block 514 may be determined prior to beginning the hearing test or prior to processing the audio signal source at block 508. In such aspects, the action of block 520 may include instructing which audio signal source should be used to conduct the hearing test to avoid negative effects of the estimated noise.

As used above, it should be understood that meeting the threshold can indicate a negative or positive inference. For example, meeting the threshold may indicate that the estimated noise level was greater than or less than the threshold. When the estimated noise level is greater than the threshold, a negative inference is indicated. When the estimated noise level is less than the threshold, a positive inference is indicated. When the estimated noise level is equal to the threshold, either a positive or negative inference is indicated.

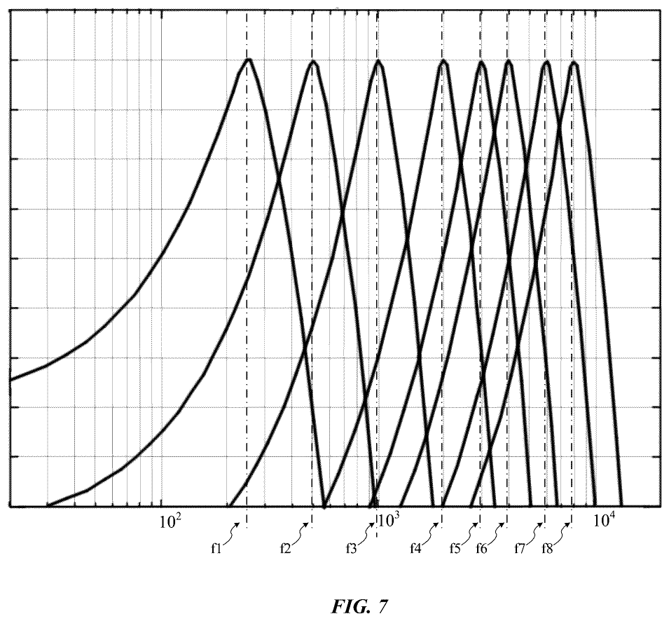

FIG. 7 illustrates an example auditory filter which can be used for evaluating the estimated noise. Although particular center frequencies are illustrated, it should be understood that any center frequencies can be used. The estimated noise can be evaluated by auditory filtering using the filter shape of FIG. 7 or one like it. Thus, the resulting evaluated noise will indicate only the levels corresponding to the frequency components which fall under the curves of FIG. 7. FIG. 7 illustrates eight center frequencies f1, f2, f3, f4, f5, f6, f7, f8. As illustrated, f3, for example corresponds to 1000 kHz. The curves centered on each of these frequencies may correspond, for example, to the test tones used for the hearing test. When a test tone is provided for the hearing test, if the estimated noise is near the same frequency as the test tone, the estimated noise can mask the test tone.

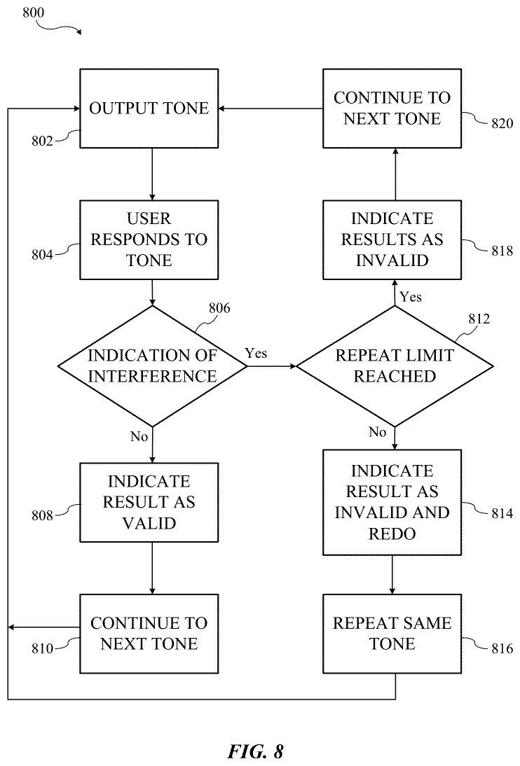

FIG. 8 illustrates a flow diagram of an example process 800 for a hearing test process, in accordance with some implementations. For explanatory purposes, the process 800 is described with respect to the electronic device 130 of FIG. 1, such as a user device. However, the process 800 is not limited to the electronic device 100 of FIG. 1 or 2, and one or more blocks (or operations) of the process 800 may be performed by one or more other components and other suitable devices. Further for explanatory purposes, the blocks of the process 800 are described herein as occurring in serial, or linearly. However, multiple blocks of the process 800 may occur in parallel. In addition, the blocks of the process 800 need not be performed in the order shown and/or one or more blocks of the process 800 need not be performed and/or can be replaced by other operations.

In the example of FIG. 8, a user device (e.g., electronic device 130) may be used to conduct a hearing test utilizing an audio device (e.g., electronic device 100) such as earbuds or headphones which may be equipped with an ANC mode. At block 802, a tone is output. The user device can provide the tone as an output signal of the user device to the audio device or as an instruction to the audio device to output the tone. In some implementations, the tone may be selected based on the ambient noise and/or estimated noise level at the user's eardrum. The tone may be output in one or more ears of the user, for example, on the right and/or left earbuds or headphones of the audio device.

At block 804, the user may respond to the tone or audio signal. In some implementations, a user interface is provided to take as input from the user a response indicating the tone played was heard or not heard by the user. At block 806, a decision is made as to whether an indication of interference was found while the tone was being output. This aspect is described in further detail below. At block 808, if interference was not indicated, then the result is indicated as valid, and at block 810, the next tone is output or the test is completed. At block 808, the result may be indicated as valid by storing a flag or some other indicator which indicates that the result is valid. In addition, the result may indicate whether the user detected the audio signal or not. The indicator may be stored along with other pertinent information, such as the output tone being used.

If, from block 806, an indication of interference is indicated, then block 812 determines if a repeat limit is reached. The repeat limit can be set to be a target number for the maximum number of repeats per tone and/or per test. For example, the flow can determine if the tone may be repeated or not. If the repeat limit is not reached, then the result is indicated as being invalid at block 814 and a repeat process is begun. At block 814, the result may be indicated as invalid or a failed test by storing a flag or some other indicator which indicates that the result is invalid or is a failed test. The indicator may be stored along with other pertinent information, such as the output tone being used and interference type and/or level. The repeat limit corresponds to a number of allowed attempts for responding to the test sound and may be any number of adjustable or set times that the test is allowed to be repeated. At block 816, the same tone is repeated by continuing to the block 802 to output the tone and get a response from a user at block 804, and so forth. Returning to block 812, if the repeat limit is reached, then the result is indicated as invalid at block 818, then at block 820 the next tone is used, which is output at block 802, or the test may be concluded and an indication made that the test failed, including which test tone was being output when the test failed.

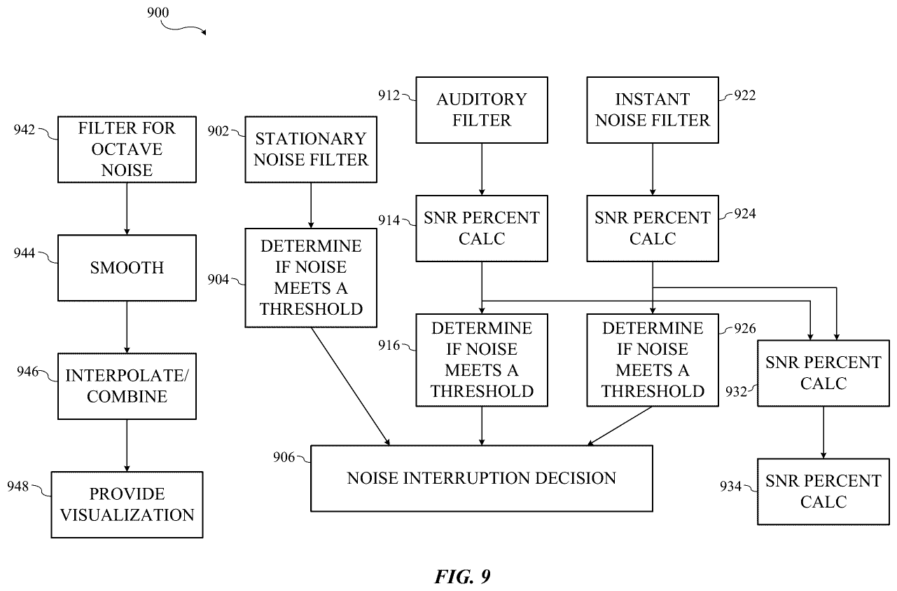

FIG. 9 illustrates a flow diagram of an example process 900 for calculating a noise interruption decision, in accordance with some implementations. This example process 900 may be used for determining if an estimated noise level at a user's eardrums interferes with an audio test, such as utilized in block 806 of FIG. 8. The process 900 may be executed on the audio device (e.g., electronic device 100) or the user device (e.g., electronic device 130). For explanatory purposes, the process 900 is described with respect to the electronic device 130 of FIG. 1, such as a user device. However, the process 900 is not limited to the electronic device 130 of FIG. 1 or 2, and one or more blocks (or operations) of the process 900 may be performed by one or more other components and other suitable devices. Further for explanatory purposes, the blocks of the process 900 are described herein as occurring in serial, or linearly. However, multiple blocks of the process 900 may occur in parallel. In addition, the blocks of the process 900 need not be performed in the order shown and/or one or more blocks of the process 900 need not be performed and/or can be replaced by other operations.

At blocks 902, 912, 922, and 942 the estimated noise, ambient noise (e.g., received via reference mic 116), and/or environmental noise (e.g., received via error mic 118) is filtered for different noise types and/or levels. At block 902, a filter for ambient stationary noise may be applied. Stationary noise is noise which is relatively constant over time. It can have a wide range of frequencies associated with it. It can include background noises like noises generated from ceiling fans, motors, electronics, and so forth. At block 904, it can be determined if the filtered noise meets a threshold. For example, the filtered noise can be compared to a model noise threshold signal or to a threshold energy level. At block 906, a noise interruption decision is made, for example, if the noise meets the threshold, then it can be determined that the physical environment surrounding the user is too noisy to conduct the test or continue the test or to record a valid result for the test tone.

At block 912, an auditory filter may be applied, such as discussed above. At block 914, a signal to noise ratio (SNR) percentage can be calculated to determine an amount of contributory auditory noise from the audio components utilized in the hearing test, including, for example digital to analog converters, analog to digital converters, speaker hiss, and so forth. This can be understood as the baseline noise of the system. In quieter environments, the SNR percentage will have a greater impact on the overall noise profile. As such, it can be calculated to determine if its contribution can affect the hearing test. In some implementations, the SNR percentage can be considered to be part of the estimated noise, as discussed above. At block 916, it can be determined if the filtered noise meets a threshold. For example, the filtered noise can be compared to a model noise threshold signal or to a threshold energy level. At block 906, a noise interruption decision is made, for example, if the noise meets the threshold, then it can be determined that the physical environment surrounding the user is too noisy to conduct the test or continue the test or to record a valid result for the test tone.

At block 922, a filter for instant or ambient instantaneous noise may be applied. Instantaneous noise is noise which is sudden and short lived. It can have a wide range of frequencies associated with it. It can include any sudden noises, like a sneeze, cough, clap, squeak, yell, doorbell, and so forth. At block 924, a signal to noise ratio (SNR) percentage can be calculated to determine an amount of contributory instant noise from the audio components utilized in the hearing test. In quieter environments, the SNR percentage will have a greater impact on the overall noise profile. As such, it can be calculated to determine if its contribution can affect the hearing test. In some implementations, the SNR percentage can be considered to be part of the estimated noise, as discussed above. At block 926, it can be determined if the filtered noise meets a threshold. For example, the filtered noise can be compared to a model noise threshold signal or to a threshold energy level. At block 906, a noise interruption decision is made, for example, if the noise meets the threshold, then it can be determined that the physical environment surrounding the user is too noisy to conduct the test or continue the test or to record a valid result for the test tone.

At block 932, the auditory filter from block 912 (by way of block 914) may be utilized along with the filtered instant noise from block 922 (by way of block 924) to determine a tone interference decision. For example, if the noise interruption decision of block 906 is found for the auditory filter noise or instant noise, then they each may cause interference in different ways. In the case of an auditory filter noise, the type of interference is a masking-type interference. The auditory filter noise can simply cover up or confuse the hearing test tone. For example, the human ear needs a separation between two different frequencies to be able to distinguish them as distinct sounds. Otherwise, they may combine and sound garbled or pulsing. An auditory filter interference near the frequency of the test tone can thus interfere with the user's ability to recognize the test tone. In the case of instant noise, the type of interference is a distraction-type interference. The sudden noise can simply distract the user from being able to recognize the hearing test tone. At block 932, the type of interference can be recorded along with the indication that an interference occurred, such as discussed above with respect to block 806 of process 800.

At block 934, the test tone can be repeated, recorded, or advanced to a new tone, such as described above with respect to the process 800.

At block 942, an octave filter can be applied to the noise. The octave filter breaks the noise down in to frequency ranges to provide total energy levels at different frequency ranges. The octave filter can be analogized to an equalizer function on a stereo, which can set levels of sound output for different frequency ranges, however, instead of setting levels, it shows what the levels are in the estimated noise. At block 944, the output of the filter can be smoothed to make the transitions between frequency ranges less severe. At block 946, the output of the filter can be interpolated or combined to estimate levels at frequencies ranges which are extracted from another frequency range. For example, a frequency range can be split into two contiguous frequency ranges and a level value applied to both which are interpolated based on the next frequency range level in either direction. In another example, a frequency range can be combined with another frequency range to reduce the number of frequency ranges represented.

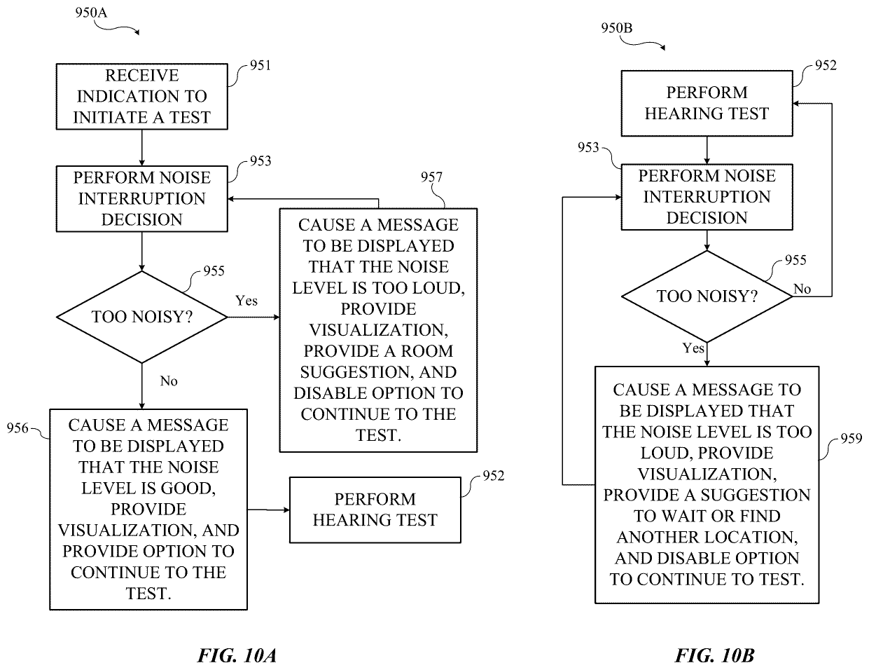

At block 948, the frequency ranges can be provided in a hearing test application to the user as a visualization of the estimated noise. A sample interface 950 is provided in FIG. 10 and a sample visualization of the estimated noise is provided by element 948v.

FIGS. 10A and 10B illustrates flow diagrams of an example process 950A and example process 950B for performing a hearing test and providing instructions based on a noise interruption decision, in accordance with some implementations. The example process 950A may be used for determining if the user's environment is quiet enough for performing a hearing test, for example, prior to the hearing test beginning. The example process 950B may be used during a hearing test to determine if the hearing test should be interrupted. The processes 950A and/or 950B may be executed on the audio device (e.g., electronic device 100) or the user device (e.g., electronic device 130). Further for explanatory purposes, the blocks of the process 950A and/or 950B are described herein as occurring in serial, or linearly. However, multiple blocks of the processes 950A and/or 950B may occur in parallel. In addition, the blocks of the processes 950A and/or 950B need not be performed in the order shown and/or one or more blocks of the processes 950A and/or 950B need not be performed and/or can be replaced by other operations.

Referring to FIG. 10A, at block 951, an indication is received to initiate a hearing test. The indication may be received, for example, on an interface of a user device (e.g., electronic device 130) by a user interacting with the interface. In some aspects, the indication may be received by the audio device (e.g., electronic device 100), for example, from the electronic device 130. At block 953, a noise interruption decision is performed. The noise interruption decision may be performed, for example, according to the block 906 of FIG. 9 and/or the blocks 514, 518, and 520 of FIG. 5 and/or as elsewhere described herein based on an estimated noise level. At block 955, if it is too noisy (e.g., the estimated noise level is above a threshold and/or, the outcome of the noise interruption decision of block 906 indicates a stationary noise level or instantaneous noise level or auditory filter noise type which would cause interference), then the flow will continue to block 957, otherwise to block 955.

At block 956, when not too noisy, a message can be caused to be displayed that indicates that the noise level is acceptable. A visualization of the noise may also be provided, such as an octave band visualization. The visualization can include color coding, such as green color-coding to indicate an acceptable result. The octave band visualization can provide vertical bars for different noise frequency ranges, arranged side-by-side for contiguous frequency ranges. The length of the vertical bars can indicate the amount of noise in each of the frequency ranges. An option can be provided to continue to the test, for example. At block 952, the hearing test can be performed upon receiving an indication from the user to continue to the test.

At block 957, when too noisy, a message can be caused to be displayed that indicates that the noise level is too high. A visualization of the noise may also be provided, similar to that described above. The visualization may be color coded, for example, with a red color, to indicate a noisy environment. A further message can be caused to be displayed that provides suggestions to obtain a quieter environment, such as switching rooms, turning off noise producing devices, and so forth. An option to continue to the test can be disabled or not shown to the user. Flow can continue back to block 953 to perform a noise interruption decision again, for example, until the user finds a quieter environment.

Referring to FIG. 10B, at block 952, a hearing test is being performed, e.g., for example, from block 952 of FIG. 10A. While performing the hearing test, at block 953, a noise interruption decision is performed. The noise interruption decision may be performed, for example, according to the block 906 of FIG. 9 and/or the blocks 514, 518, and 520 of FIG. 5 and/or as elsewhere described herein based on an estimated noise level. At block 955, if it is too noisy (e.g., the estimated noise level is above a threshold and/or, the outcome of the noise interruption decision of block 906 indicates a stationary noise level or instantaneous noise level or auditory filter noise type which would cause interference), then the flow will continue to block 959, otherwise to block 952, to continue to perform the hearing test.

At block 959, when too noisy, a message can be caused to be displayed that indicates that the noise level is too high. A visualization of the noise may also be provided, similar to that described above. The visualization may be color coded, for example, with a red, yellow, or orange color, to indicate a noisy environment. A further message can be caused to be displayed that provides a suggestion to wait for the noise to subside (e.g., in the case of an interference caused by instantaneous noise or output of an auditory filter) or a suggestion to find another quieter location to continue to the test. While too noisy, an option to continue the test can be disabled. The flow can continue to block 953 to loop back to perform a noise interruption decision. When the noise issue is resolved, the option to continue the test may be enabled, and the flow can continue back to block 952 to continue to perform the hearing test.

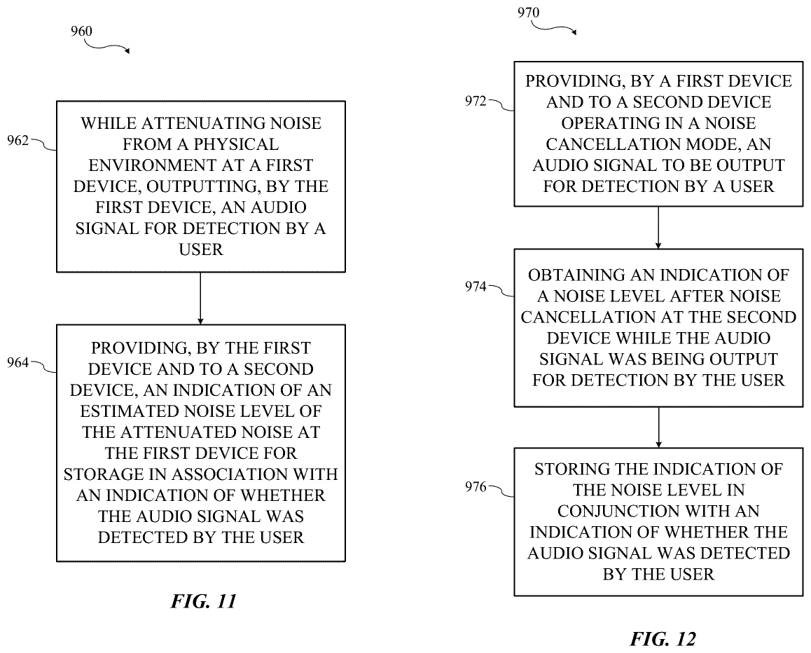

FIG. 11 illustrates a flow diagram of an example process 960 for estimating a noise level of the ambient noise, in accordance with some implementations. The process 960 may be executed on the audio device (e.g., electronic device 100). For explanatory purposes, the blocks of the process 960 are described herein as occurring in serial, or linearly. However, multiple blocks of the process 960 may occur in parallel. In addition, the blocks of the process 960 need not be performed in the order shown and/or one or more blocks of the process 960 need not be performed and/or can be replaced by other operations.

At block 962, while attenuating noise from a physical environment at a first device, outputting, by the first device, an audio signal for detection by a user. Noise from the physical environment can be attenuated by ANC, by PNC blocking with the device bode and/or device sealing member, or combinations thereof. The audio signal output can be a hearing test tone as described above or other types of audio signal output. At block 964, an indication of an estimated noise at the first device is provided, by the first device to a second device, for storage in association with an indication of whether the audio signal was detected by the user. The indication of the estimated noise level can be a noise intensity value, a noise filter or characterization result, a result from a comparison of the estimated noise level to a threshold, or the like, in accordance with implementations described herein. Storage can be in the memory of the first device and/or the second device. The indication of whether the audio signal was detected by the user can be stored in the first device and/or the second device.

In one or more implementations, implementations may include one or more of the following features. The audio signal may be a first audio signal, and when the estimated noise level of the attenuated noise satisfies a threshold, a second audio signal is output, by the first device, for detection by the user, where the first audio signal and the second audio signal have different frequencies. In one or more implementations, when the estimated noise level of the attenuated noise does not satisfy a threshold and when a number of attempts for outputting the audio signal is below a target number, the audio signal is output, by the first device, again for detection by the user. In one or more implementations, the first device may include an audio input device, an audio output device, and a sealing member between the audio input device and the audio output device; and the estimated noise level of the attenuated noise is based at least in part on an estimated performance of the sealing member. In one or more implementations, prior to outputting the audio signal, a test is executed to obtain the estimated performance of the sealing member. In one or more implementations, the noise includes ambient stationary noise and ambient instantaneous noise. In one or more implementations, the audio signal corresponds to a hearing test tone. In one or more implementations, the noise is attenuated at a location between the first device and an ear of the user. In one or more implementations, the estimated noise level of the attenuated noise at the first device represents an estimated attenuated noise level at an eardrum of the user.

FIG. 12 illustrates a flow diagram of an example process 970 for outputting an audio signal for detection by a user, in accordance with some implementations. For explanatory purposes, the process 970 is described with respect to the user device (e.g., electronic device 130) of FIG. 1. However, the process 970 is not limited to the electronic device 130 of FIGS. 1-3, and one or more blocks (or operations) of the process 970 may be performed by one or more other components and other suitable devices. Further, for explanatory purposes, the blocks of the process 970 are described herein as occurring in serial, or linearly. However, multiple blocks of the process 970 may occur in parallel. In addition, the blocks of the process 970 need not be performed in the order shown and/or one or more blocks of the process 970 need not be performed and/or can be replaced by other operations.

At block 972, an audio signal is provided, by a first device and to a second device operating in a noise cancellation mode, to be output for detection by a user. The audio signal can be a signal sent for playback to earbuds or headphones, such as electronic device 100. The audio signal can be a hearing test tone which is to be detected by a user. At block 974, an indication can be obtained of a noise level after noise cancellation at the second device while the audio signal was being output for detection by the user. The indication of the estimated noise level can be a noise intensity value, a noise filter or characterization result, a result from a comparison of the estimated noise level to a threshold, or the like, in accordance with implementations described herein. At block 976, the indication of the noise level can be stored in conjunction with an indication of whether the audio signal was detected by the user. The indication of whether the audio signal was detected by the user can be stored in the first device and/or the second device.

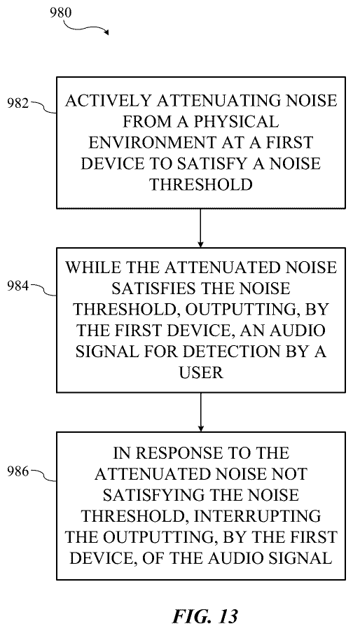

FIG. 13 illustrates a flow diagram of an example process 980 for outputting an audio signal for detection by a user while actively attenuating noise from a physical environment at a first device, in accordance with some implementations. For explanatory purposes, the process 980 is described with respect to the audio device (e.g., electronic device 100) of FIG. 1. However, the process 980 is not limited to the electronic device 100 of FIGS. 1-3, and one or more blocks (or operations) of the process 980 may be performed by one or more other components and other suitable devices. Further, for explanatory purposes, the blocks of the process 980 are described herein as occurring in serial, or linearly. However, multiple blocks of the process 980 may occur in parallel. In addition, the blocks of the process 980 need not be performed in the order shown and/or one or more blocks of the process 970 need not be performed and/or can be replaced by other operations.

At block 982, noise from a physical environment at a first device is actively attenuated to satisfy a noise threshold. For example, ANC can be used to actively attenuate noise from the physical environment to reduce the noise at the user's eardrum so that the estimated noise level at the user's eardrum meets a noise threshold. This can include one or more thresholds including a threshold for instantaneous noise, a threshold for continuous noise, and a threshold for an output of an auditory filter. At block 984, while the attenuated noise satisfies the noise threshold, outputting, by the first device, and audio signal for detection by a user. The audio signal can be a signal sent for playback to earbuds or headphones, such as electronic device 100. The audio signal can be a hearing test tone which is to be detected by a user. At block 986, the attenuated noise may not satisfy the one or more thresholds noted above. In response to the attenuated noise not satisfying the one or more noise thresholds, the outputting of the audio signal by the first device is interrupted. Thus, the noise level increasing can cause the testing to stop by way of an interruption. An indication of the interruption can be stored along with information regarding the audio signal. The indication, for example, can indicate which threshold of the one or more thresholds was exceeded and an interference-type (e.g., distraction or masking) associated with the interruption.

In one or more implementations, implementations may include one or more of the following features. In one or more implementations, obtaining the indication of the noise level after noise cancellation at the second device may include receiving, by the first device from the second device, the indication of the noise level after noise cancellation. In one or more implementations, obtaining the indication of the noise level after noise cancellation at the second device may include: receiving a second audio signal from the second device; and estimating the noise level on the first device based on the second audio signal from the second device. In one or more implementations, an input may be received, by the first device, from the user indicating that whether the audio signal was detected by the user. In one or more implementations, frequency characteristics of the noise level may be modeled, by the first device, after noise cancellation, and the audio signal provided is selected based on the frequency characteristics of the noise level. In one or more implementations, when the indication of the noise level after noise cancellation does not satisfy a threshold, an indication is stored that the noise level did not satisfy the threshold in conjunction with the audio signal. In one or more implementations, when a number of attempts for providing the audio signal is below a target number, the audio signal is provided again, by the first device and to the second device operating in the noise cancellation mode, for detection by the user, and when the number of attempts for providing the audio signal satisfies the target number, an indication of a failed test is stored for the audio signal. In one or more implementations, when the indication of the noise level after noise cancellation does not satisfy the threshold based on an instantaneous noise in a physical environment of the second device, an indication is stored that the noise level did not satisfy the threshold based on a distraction-type noise; and when the indication of the noise level after noise cancellation does not satisfy the threshold based on an auditory filter of noise in the physical environment of the second device, an indication is stored that the noise level did not satisfy the threshold based on a masking-type noise. In one or more implementations, the threshold is determined based on a frequency characteristic of the noise level after noise cancellation and on a frequency characteristic of the audio signal. In one or more implementations, when the indication of the noise level after noise cancellation does not satisfy a threshold, prior to providing the audio signal, wait until the indication of the noise level after noise cancellation satisfies the threshold.

As described above, one aspect of the present technology is the gathering and use of data available from specific and legitimate sources for providing automatic adaptive noise cancellation for electronic devices. The present disclosure contemplates that in some instances, this gathered data may include personal information data that uniquely identifies or can be used to identify a specific person. Such personal information data can include voice data, demographic data, location-based data, online identifiers, telephone numbers, email addresses, home addresses, data or records relating to a user's health or level of fitness (e.g., vital signs measurements, medication information, exercise information), date of birth, or any other personal information.

The present disclosure recognizes that the use of such personal information data, in the present technology, can be used to the benefit of users. For example, the personal information data can be used for operating an electronic device to provide automatic adaptive noise cancellation for electronic devices. Accordingly, use of such personal information data may facilitate transactions (e.g., on-line transactions). Further, other uses for personal information data that benefit the user are also contemplated by the present disclosure. For instance, health and fitness data may be used, in accordance with the user's preferences to provide insights into their general wellness, or may be used as positive feedback to individuals using technology to pursue wellness goals.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominently and easily accessible by users, and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations which may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of operating an electronic device to provide automatic adaptive noise cancellation for electronic devices, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing identifiers, controlling the amount or specificity of data stored (e.g., collecting location data at city level rather than at an address level), controlling how data is stored (e.g., aggregating data across users), and/or other methods such as differential privacy.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data.

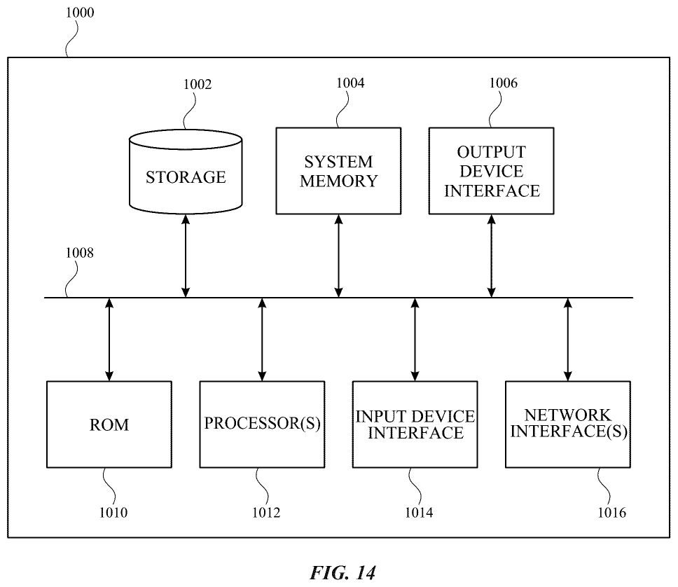

FIG. 14 illustrates an electronic system 1000 with which one or more implementations of the subject technology may be implemented. The electronic system 1000 can be, and/or can be a part of, one or more of the electronic devices 100 and/or 130 shown in FIGS. 1-3. The electronic system 1000 may include various types of computer readable media and interfaces for various other types of computer readable media. The electronic system 1000 includes a bus 1008, one or more processing unit(s) 1012, a system memory 1004 (and/or buffer), a ROM 1010, a permanent storage device 1002, an input device interface 1014, an output device interface 1006, and one or more network interfaces 1016, or subsets and variations thereof.

The bus 1008 collectively represents all system, peripheral, and chipset buses that communicatively connect the numerous internal devices of the electronic system 1000. In one or more implementations, the bus 1008 communicatively connects the one or more processing unit(s) 1012 with the ROM 1010, the system memory 1004, and the permanent storage device 1002. From these various memory units, the one or more processing unit(s) 1012 retrieves instructions to execute and data to process in order to execute the processes of the subject disclosure. The one or more processing unit(s) 1012 can be a single processor or a multi-core processor in different implementations.

The ROM 1010 stores static data and instructions that are needed by the one or more processing unit(s) 1012 and other modules of the electronic system 1000. The permanent storage device 1002, on the other hand, may be a read-and-write memory device. The permanent storage device 1002 may be a non-volatile memory unit that stores instructions and data even when the electronic system 1000 is off. In one or more implementations, a mass-storage device (such as a magnetic or optical disk and its corresponding disk drive) may be used as the permanent storage device 1002.

In one or more implementations, a removable storage device (such as a flash drive) may be used as the permanent storage device 1002. Like the permanent storage device 1002, the system memory 1004 may be a read-and-write memory device. However, unlike the permanent storage device 1002, the system memory 1004 may be a volatile read-and-write memory, such as random access memory. The system memory 1004 may store any of the instructions and data that one or more processing unit(s) 1012 may need at runtime. In one or more implementations, the processes of the subject disclosure are stored in the system memory 1004, the permanent storage device 1002, and/or the ROM 1010. From these various memory units, the one or more processing unit(s) 1012 retrieves instructions to execute and data to process in order to execute the processes of one or more implementations.

The bus 1008 also connects to the input and output device interfaces 1014 and 1006. The input device interface 1014 enables a user to communicate information and select commands to the electronic system 1000. Input devices that may be used with the input device interface 1014 may include, for example, alphanumeric keyboards and pointing devices (also called “cursor control devices”). The output device interface 1006 may enable, for example, the display of images generated by electronic system 1000. Output devices that may be used with the output device interface 1006 may include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more implementations may include devices that function as both input and output devices, such as a touchscreen. In these implementations, feedback provided to the user can be any form of sensory feedback, such as visual feedback, auditory feedback, or tactile feedback; and input from the user can be received in any form, including acoustic, speech, or tactile input.

Finally, as shown in FIG. 14, the bus 1008 also couples the electronic system 1000 to one or more networks and/or to one or more network nodes, through the one or more network interface(s) 1016. In this manner, the electronic system 1000 can be a part of a network of computers (such as a LAN, a wide area network (“WAN”), or an Intranet, or a network of networks, such as the Internet. Any or all components of the electronic system 1000 can be used in conjunction with the subject disclosure.

Implementations within the scope of the present disclosure can be partially or entirely realized using a tangible computer-readable storage medium (or multiple tangible computer-readable storage media of one or more types) encoding one or more instructions. The tangible computer-readable storage medium also can be non-transitory in nature.

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device, e.g., via one or more wired connections, one or more wireless connections, or any combination thereof.

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data. Computer-executable instructions also can be organized in any format, including routines, subroutines, programs, data structures, objects, modules, applications, applets, functions, etc. As recognized by those of skill in the art, details including, but not limited to, the number, structure, sequence, and organization of instructions can vary significantly without varying the underlying logic, function, processing, and output.

While the above discussion primarily refers to microprocessor or multi-core processors that execute software, one or more implementations are performed by one or more integrated circuits, such as ASICs or FPGAs. In one or more implementations, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

As used in this specification and any claims of this application, the terms “base station”, “receiver”, “computer”, “server”, “processor”, and “memory” all refer to electronic or other technological devices. These terms exclude people or groups of people. For the purposes of the specification, the terms “display” or “displaying” means displaying on an electronic device.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The predicate words “configured to”, “operable to”, and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration”. Any embodiment described herein as “exemplary” or as an “example” is not necessarily to be construed as preferred or advantageous over other implementations. Furthermore, to the extent that the term “include”, “have”, or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for”.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

What is claimed is:

1. A method, comprising:
while attenuating noise from a physical environment at a first device, outputting, by the first device, an audio signal for detection by a user; and
providing, by the first device and to a second device, an indication of an estimated noise level of the attenuated noise at the first device for storage in association with an indication of whether the audio signal was detected by the user.

2. The method of claim 1, wherein the audio signal is a first audio signal, further comprising: when the estimated noise level of the attenuated noise satisfies a threshold, outputting, by the first device, a second audio signal for detection by the user, wherein the first audio signal and the second audio signal have different frequencies.

3. The method of claim 1, further comprising: when the estimated noise level of the attenuated noise does not satisfy a threshold and when a number of attempts for outputting the audio signal is below a target number, outputting, by the first device, the audio signal again for detection by the user.

4. The method of claim 1, wherein the first device comprises an audio input device, an audio output device, and a sealing member between the audio input device and the audio output device, and wherein the estimated noise level of the attenuated noise is based at least in part on an estimated performance of the sealing member.

5. The method of claim 4, further comprising: prior to outputting the audio signal, executing a test to obtain the estimated performance of the sealing member.

6. The method of claim 1, wherein the noise includes ambient stationary noise and ambient instantaneous noise.

7. The method of claim 1, wherein the audio signal corresponds to a hearing test tone.

8. The method of claim 1, wherein the noise is attenuated at a location between the first device and an ear of the user.

9. The method of claim 1, wherein the estimated noise level of the attenuated noise at the first device represents an estimated attenuated noise level at an eardrum of the user.

10. A method comprising:
providing, by a first device and to a second device operating in a noise cancellation mode, an audio signal to be output for detection by a user;
obtaining an indication of a noise level after noise cancellation at the second device while the audio signal was being output for detection by the user; and
storing the indication of the noise level in conjunction with an indication of whether the audio signal was detected by the user.

11. The method of claim 10, wherein obtaining the indication of the noise level after noise cancellation at the second device comprises receiving, by the first device from the second device, the indication of the noise level after noise cancellation.

12. The method of claim 10, wherein obtaining the indication of the noise level after noise cancellation at the second device comprises:
receiving a second audio signal from the second device; and
estimating the noise level on the first device based on the second audio signal from the second device.

13. The method of claim 10, further comprising:
receiving, by the first device, an input from the user indicating that whether the audio signal was detected by the user.

14. The method of claim 10, further comprising:
modeling, by the first device, frequency characteristics of the noise level after noise cancellation; and
selecting the audio signal provided based on the frequency characteristics of the noise level.

15. The method of claim 10, further comprising:
when the indication of the noise level after noise cancellation does not satisfy a threshold, storing an indication that the noise level did not satisfy the threshold in conjunction with the audio signal.

16. The method of claim 15, further comprising:
when a number of attempts for providing the audio signal is below a target number, providing, by the first device and to the second device operating in the noise cancellation mode, the audio signal again for detection by the user; and
when the number of attempts for providing the audio signal satisfies the target number, storing an indication of a failed test for the audio signal.

17. The method of claim 15, further comprising:
when the indication of the noise level after noise cancellation does not satisfy the threshold based on an instantaneous noise in a physical environment of the second device, storing an indication that the noise level did not satisfy the threshold based on a distraction-type noise; and
when the indication of the noise level after noise cancellation does not satisfy the threshold based on an auditory filter of noise in the physical environment of the second device, storing an indication that the noise level did not satisfy the threshold based on a masking-type noise.

18. The method of claim 15, wherein the threshold is determined based on a frequency characteristic of the noise level after noise cancellation and on a frequency characteristic of the audio signal.

19. The method of claim 10, further comprising:
when the indication of the noise level after noise cancellation does not satisfy a threshold, prior to providing the audio signal, waiting until the indication of the noise level after noise cancellation satisfies the threshold.

20. A system, comprising:
a processor; and
a memory device containing instructions which, when executed by the processor, cause the processor to:
actively attenuating noise from a physical environment at a first device to satisfy a noise threshold;
while the attenuated noise satisfies the noise threshold, outputting, by the first device, an audio signal for detection by a user; and
in response to the attenuated noise not satisfying the noise threshold, interrupting the outputting, by the first device, of the audio signal.
